(19) United States
(12) Patent Application Publication
(10) Pub. No.: US 2015/0217121 A1
(43) Pub. Date: Aug. 6, 2015

(54) METHOD AND SYSTEM FOR A WEARABLE DEFIBRILLATOR

(71) Applicants: SANDYA SUBRAMANIAN, GRAND RAPIDS, MI (US); POWEI KANG, ROCHESTER HILLS, MI (US); CHUN-MING SO, MERCER ISLAND, WA (US); MELINDA CHEN, LUTHERVILLE, MD (US); TAYLOR LAM, THOUSAND OAKS, CA (US); TODD J. COHEN, PORT WASHINGTON, NY (US)

(72) Inventors: SANDYA SUBRAMANIAN, GRAND RAPIDS, MI (US); POWEI KANG, ROCHESTER HILLS, MI (US); CHUN-MING SO, MERCER ISLAND, WA (US); MELINDA CHEN, LUTHERVILLE, MD (US); TAYLOR LAM, THOUSAND OAKS, CA (US); TODD J. COHEN, PORT WASHINGTON, NY (US)

(21) Appl. No.: 14/600,576

(22) Filed: Jan. 20, 2015

Related U.S. Application Data
(60) Provisional application No. 61/929,464, filed on Jan. 20, 2014.

(51) Int. Cl. A61N 1/39; A61N 1/365

(52) U.S. Cl. CPC A61N 1/3968; A61N 1/3993; A61N 1/3987; A61N 1/365

(57) ABSTRACT

A method and system for an improved wearable defibrillator specifically optimized for patient comfort and compliance. The system is capable of sensing information about the patient, determining whether the patient is experiencing a life-threatening arrhythmia, and if so, deliver life-saving defibrillation. The system is specifically designed, constructed, and configured to allow for minimal weight on the shoulders and its associated lumbar spine compression when standing and minimal pressure on the abdomen or thorax when supine in order to increase patient comfort and compliance. This configuration and design can be implemented in several forms including but not limited to the material(s) used, the placement of various components, the durability of the components, the operative or communicative connection between various components, the efficiency and/or accuracy of the various components in performing their functions, and/or the maintenance procedure.

### CROSS-REFERENCE TO RELATED PATENT APPLICATION

This patent application is based upon and claims the benefit of the priority of U.S. Provisional Patent Application Ser. No. 61/929,464, filed Jan. 20, 2014, which is incorporated herein by reference in its entirety.

### FIELD OF THE INVENTION

This invention relates to wearable devices used for detecting and treating cardiac arrhythmias through defibrillation. More particularly, the invention relates to wearable defibrillators that have additional functions and are comfortable to wear.

### BACKGROUND OF THE INVENTION

Sudden cardiac arrest (SCA) is a leading killer in the United States and in the rest of the world as well. SCA occurs when there is a ventricular arrhythmia, an electrical problem with the heart, which causes it to stop pumping blood to the body. Although the causes vary, SCA is the leading cause of death in the U.S. with a 95% fatality rate. Each year about 450,000 people in the U.S. die from SCA, and 340,000 of these are caused by ventricular arrhythmias. As Americans age, these numbers are expected to rise. Approximately 80% of arrhythmias occur in people who are 45+, and by 2030 20% of the population will be over the age of 65 (up from 13%). The unpredictability of SCA increases the risk to the patient; 80-88% of SCAs occur at home where medical personnel are not available, and 40% are not witnessed at all. Patients experiencing SCA quickly lose consciousness and are likely to die if they do not receive treatment within minutes. Therefore, automatic administration of treatment at the first sign of an irregular heartbeat is crucial.

The normal treatment is to defibrillate the heart by sending a high energy shock through the heart. This resets the electrical activity in the heart, hopefully resetting the heart to normal sinus rhythm. The long-term solution is an implantable cardioverter-defibrillator (ICD), which detects abnormal sinus rhythms and shocks the heart when the heart needs to be defibrillated. By virtue of direct implantation over the heart, the ICD can bypass the impedance of the skin to deliver life-saving defibrillating shocks. However, there is a three-month waiting period for an ICD due to legal and regulatory reasons during which patients are still vulnerable to SCA. The current standard of care for patients during this waiting period is the wearable cardioverter-defibrillator (WCD), which externally monitors the patient's ECG at all times and can provide defibrillation when needed.

Wearable cardioverter-defibrillator devices used to detect and treat cardiac arrhythmias are known. The only WCD currently on the market is the Zoll's LifeVest WCD. U.S. Pat. No. 4,928,690 is Zoll's original patent for a wearable cardioverter-defibrillator. Subsequent designs of WCDs and related devices with additional features and improvements have given rise to more recent patents in this field. U.S. Pat. No. 8,369,944 describes a wearable cardioverter-defibrillator with audio input and output capabilities. Additional relevant publications include U.S. Pat. No. 8,185,199, which describes a method of monitoring physiological signals during electrical stimulation, and PCT Publication No. WO 2012/082547, which refers to an accessory kit for a wearable defibrillator to make the device waterproof.

Although the prior art includes various additional modifications to wearable defibrillators, none are specifically optimized to increase patient comfort and compliance, specifically with respect to the posture and configuration of the various components and their effects on lumbar spine compression and pressure on the abdomen or thorax. In addition, external sensing electrodes in the current Zoll LifeVest WCD is very uncomfortable to the patient. Further, these external systems may lose contact and/or move on the surface of the skin, creating noise, and causing the device to alarm prematurely.

A team of biomedical engineering students at the Johns Hopkins University called “CardioGuard”, led by Dr. Todd Cohen, scientifically evaluated patients and their experience with the currently used Zoll LifeVest defibrillator. They identified specific areas of patient dissatisfaction which contribute to noncompliance. Specifically, the results of their research demonstrated that the assembly and maintenance of the current device is relatively complex, the electrodes are uncomfortable and create noise, and the large shoulder harness box that serves as the patient's user interface is uncomfortable and asymmetric and can be bulky and disruptive during sleep.

### SUMMARY OF THE INVENTION

The CardioGuard team then proceeded to develop a novel wearable defibrillator system, the subject of this application, which they called the “QRSTee”. The name illustrates the simplicity of the system, since it is closer to a Tee shirt in weight and placement than the complicated harness-like device made by Zoll.

The current invention described herein creates a lower profile self-contained wearable defibrillator with minimal, if any, assembly. Sensing is optimized with either long-term adhesive electrodes, textile electrodes, or wireless transmission from an implantable cardiac monitor similar to the injectable Medtronic Reveal LINQ device. The later innovation is a breakthrough since the sensing in the current Zoll device creates lots of noise and patients often have to abort alerts that the device is about to shock as a result of this external electrode/skin contact noise. The wireless transmission between an implantable device and the closely coupled vest may minimize and potentially eliminate this problem. In addition, this version of the device eliminates the placement and removal/assembly of any type of electrode. Finally, the implantable cardiac monitor itself has proven very useful and recording an entire assortment of arrhythmias around the clock. The current Medtronic Reveal LINQ implantable cardiac monitor is injectable, takes seconds to implant, and is about the size of a small paper clip.

The recently developed wearable defibrillator by the Johns Hopkins University CardioGuard Team contains the defibrillator components including central processing unit, capacitors, and defibrillating electrodes. The wearable defibrillator, itself, may contain a remote monitoring system such as the Medtronic MyCareLink that communicates with the LINQ device to detect and analyze an arrhythmia to get her with the wearable defibrillator's CPU. In addition, the wearable defibrillator can communicate to a patient interface in the form of a watch.

The CardioGuard team's system improves on the ease of assembly, the comfort, the functionality, and eliminates the large separate computer/power supply box which also contains the patient interface in the form of a shoulder harness. Instead, the system can be slipped on and off, easily washed with soap and water (has water resistance), and the developed user interface for the QRSTee is a watch like device (worn on the wrist) that wirelessly communicates with the wearable cardioverter defibrillator.

The present invention provides a wearable cardioverter defibrillator which comprises one or more electrical conductors and a control system consisting of at least one power source, at least one electrical circuit including a capacitor, and at least one computer/central processing unit, all configured to increase patient comfort and compliance by minimizing lumbar spine compression when standing and pressure on the abdomen or thorax when supine. The invention may also include one or more user interfaces, one or more textile garments, one or more secondary sensors, and a waterproof, breathable, hydrophobic, or pressing-exerting material. The present invention continuously monitors user's electrocardiogram (ECG) data, and may continuously monitor secondary information from the patient such as, but not limited to, heart rate, blood pressure, acceleration, etc. The present invention processes the user's health data and provides defibrillation if the at least one central processing unit determines the user is suffering from life-threatening arrhythmias. Additionally, a remote monitoring system similar to the Medtronic MyCareLink system can be built into the wearable defibrillator and provide around the clock secure wireless communication to “the cloud” to a remote terminal station or device. Additionally, external sensing electrodes can entirely be eliminated utilizing an implantable cardiac monitor device such as the Medtronic Reveal LINQ which already wirelessly communicates with the Medtronic MyCareLink system using Bluetooth methodology. This advance eliminates the placement, assembly, and noise (which creates artifacts and false alarms on the current device).

In a preferred embodiment of the invention, the system takes the general form of a shirt or vest and may include other accessories such as watch-like interface. The large area covered by the system provides ample space over which a variety of sensors such as, but not limited to, ECG electrodes and blood pressures sensors can be placed. Additionally, the large area provides ample space over which a variety of therapeutic devices such as, but not limited to, defibrillation electrodes can be placed. The system integrates technical components of the control system. The control system as a whole is able to analyze the electrical signal from the electrical conductors and provide heart rhythm corrective therapy if necessary. The control system may be divided into discrete components that can be integrated anywhere around the patient's body, for example, on either side of the body approximately three inches from the armpit; the system integrates the discrete components in such a way to maximize user comfort. The layout of the computerized components, defibrillator components, power supply, and remote monitor within the wearable vest are laid out in a balance and quasi symmetrical fashion in order to minimize the weight on the shoulders and lumbar spine, and minimize bulk of the system when one rolls during their sleep. The system may be fixed to a textile garment that may be made of materials which are waterproof, breathable, or easily cleanable. One version utilizes compression shirt and/or neoprene surfing material to encase the wearable defibrillator component. Another version utilizes breathable athletic materials similar to those used by athletes manufactured by Under Armour. The system is then resistant to sweat and additionally easily maintained by the user. The material may include, but is not limited to, super-hydrophobic fabric or bactericidal fabric.

The system according to the invention analyzes a patient's electrocardiogram to determine whether the patient is suffering from life-threatening arrhythmias that must be treated. The electrical conductors responsible for sensing may be made of a conductive textile material, hereby referred to as textile electrodes, which may be directly sewed into the a garment or kept in place with alternative means. The textile electrodes may cover very large areas such as around the arm or wrapping around entire torso to provide optimal signal. The textile electrodes may be incorporated with stabilizing elements to decrease noise due to movement or any external or internal noise sources. The electrical conductors for sensing may use one or more adhesive electrodes which may be long-term which are placed around the user's body. The long term adhesive electrodes may last up to but not limited to six weeks of usage. The long-term adhesive electrodes may include the electrical conductors responsible for defibrillation. In a preferred embodiment, external electrodes as were previously described, are entirely eliminated by the use of an implantable cardiac monitor which provides the electrical conductors for sensing. This device would wirelessly communicate with the wearable defibrillator. The electrocardiographic signals and heart rate related information would be transmitted to the wearable defibrillator. Additionally, a much more ergonomic and comfortable patient user interface for the QRSTee wearable defibrillator has already been developed in the form of a wrist worn device like an iWatch. This user interface can provide a point of communication to and from the patient or subject. It can also provide alerts in the form of vibration, visual, or audio forms.

The electrical conductors may include wireless technology to communicate with the device's central processing unit. The electrical conductors may contain additional electronic parts to be able to analyze and transmit data. The electrical conductors may be reusable or partly reusable (i.e., some components are disposable and others are reuseable). The electrical conductors may be made of some conductive ink material that can be printed directly on the skin of the patient or onto a fabric material.

The device may have one or more user interfaces that are operatively or communicatively connected to the control system. In the case the system detects the user is suffering from a life-threatening arrhythmia, one or more user interfaces may alert the user utilizing, not limited to, auditory, tactile, and visual feedback. One or more user interfaces may have an overriding mechanism which allows the patient to override any impending therapy. One or more of the electrical conductors may be capable of an impedance reducing mechanism which includes, but is not limited to, a gel release mechanism. The device may incorporate secondary information such as, but not limited to, heart rate, blood pressure, or movement to cross-validate ECG data. The current user interface is worn on the wrist like a watch.

Additionally, the system provides for improved sensing utilizing a subcutaneous implantable cardiac monitor which wirelessly communicates with a lightweight self-contained wearable defibrillator, thereby eliminating the noise, discomfort, and assembly required from external electrodes. In addition, the system also provides for a separate watch-like user interface (similar to an iWatch) to provide information to and from the patient. The novel wearable defibrillator also contains remote monitoring capabilities which operates around the clock with data that can be transmitted to and from “the cloud” to remote devices such as smart devices/computers/terminals.

In one embodiment of the invention, the wearable defibrillator is a single self-contained garment, which contains the defibrillator with its electrodes and its components including a computer. The system is water-resistant or waterproof and encased and can be simply washed off under a sink or shower with soap.

In another embodiment of the invention, the main wearable defibrillator can wirelessly communicate with an implantable cardiac monitoring device such as the Medtronic LINQ. The implantable device would eliminate many of the problems created by surface electrodes utilized in the currently approved Zoll LifeVest device. In addition, the LINQ already communicates with a remote monitoring system (MyCareLink) that sends information/alerts wirelessly to the patient's physician. This remote monitoring system could be built into the wearable defibrillator or operate as a separate remote station. The advantage of the former, is apparent, i.e., having the ability to always remotely monitor and transmit data anywhere, all the time.

In another embodiment of the invention, a lightweight, wearable patient interface, in the form of a wrist worn computerized device similar to a watch, has been developed for improved patient interactions including the ability to abort any insignificant device alerts/alarms and/or transmit information via the remote monitoring system.

In another embodiment of the invention, a wearable heart rhythm treatment system for treatment of a subject comprises:

a self-contained ergonomic waterproof or water-resistant wearable heart rhythm treatment device comprising at least two therapy delivering electrical conductors coupled to the subject's skin for the purpose of treating an abnormal heart rhythm;

at least two sensing electrical conductors coupled to the subject's skin to detect abnormal heart rhythms; and

a control and remote monitoring system comprising of at least one power source, at least one electrical circuit including a capacitor, and at least one computer/central processing unit capable of analyzing the electrical signal sensed by the above system and providing a heart rhythm corrective therapy through one or more of said electrical conductors to treat the heart rhythm abnormality,

wherein a user interface wirelessly communicates with the subject and provides feedback as to the status of the subject, their heart rhythm, including alerts, alarms, and necessary actions to abort said therapy, and

wherein all components of said wearable system are designed, constructed, and configured to minimize the weight of said system applied to the patient's shoulders and resultant lumbar spine compression while standing and component pressure on the abdomen or thorax while lying down or supine.

In another embodiment of a system of the invention, the sensing system uses electrical conductors that are directly attached to the wearable heart rhythm treatment device.

In another embodiment of a system of the invention, the electrical conductors are long-term adhesive electrodes.

In another embodiment of a system of the invention, the sensing electrical conductors coupled to the skin are contained in a separate system subcutaneously implanted cardiac monitoring device, which is closely coupled to the wearable heart rhythm treatment device and communicates wirelessly heart rhythm related information from the subject.

In another embodiment of a system of the invention, the control system and remote monitoring system contained within the wearable heart rhythm treatment device can wirelessly and securely send and receive information to and/or “the cloud” or other remote sites or locations regarding the wearable heart rhythm device's performance, function, arrhythmia detection and treatment, alerts, and alarms, which information is then accessible via a world wide web connected computerize system.

In another embodiment of a system of the invention, the remote monitoring system utilizes a cellular service, wifi service, and/or other wireless system to transmit and receive data.

In another embodiment of a system of the invention, the treatment therapies includes both cardioversion and defibrillation therapies.

In another embodiment of a system of the invention, the treatment therapies also include pacing therapies.

In another embodiment of a system of the invention, detecting abnormal heart rhythms is categorized into significant slow heart rhythms requiring pacing therapy and significant fast heart rhythms requiring cardioversion or defibrillation therapy.

In another embodiment of a system of the invention, the user interface is a separately wearable device that wirelessly displays and communicates information from the wearable heart rhythm device to the subject to provide information regarding their status as well as that of their wearable heart rhythm device, any arrhythmias and/or shock warnings or alerts, as well as the ability to abort a shock.

In another embodiment of a system of the invention, the user interface is a wrist worn device similar to a watch.

In another embodiment of the invention, a wearable treatment system for treatment of a subject comprises:

an ergonomic water resistant wearable heart rhythm treatment device comprising at least two therapy delivering electrical conductors coupled to the subject's skin for the purpose of treating an abnormal heart rhythm; at least two sensing electrical conductors coupled to the subject's skin for the purpose of sensing and detecting abnormal heart rhythms; a control and remote monitoring system comprising of at least one power source, at least one electrical circuit including a capacitor, and at least one computer/central processing unit capable of analyzing the electrical signal sensed by the above system and providing a heart rhythm corrective therapy through one or more of said electrical conductors in order to treat the heart rhythm abnormality; and

a user interface which wirelessly communicates with the subject and provides feedback as to the status of the subject, their heart rhythm, including alerts, alarms, and necessary actions in order to abort said therapy,

wherein all components of said wearable system are designed, constructed, and configured to minimize the weight of said system applied to the patient's shoulders and resultant lumbar spine compression while standing; and component pressure on the abdomen or thorax while lying down or supine.

In another embodiment of a system of the invention, part of the control system, sensing electrodes, and/or power supply are accessible and/or removable by the subject and the point of detachment/reattachment is designed to keep out water.

In another embodiment of a system of the invention, one or more of the electrical conductors is made from a conductive textile material or woven into a breathable conductive fabric.

In another embodiment of a system of the invention, the electrical conductors are secured in place with one or more stabilizing elements to decrease noise.

In another embodiment of a system of the invention, one or more of the electrical conductors and/or the control system are fully enclosed between two layers of textile and not readily accessible to the subject.

In another embodiment of a system of the invention, one or more of the electrical conductors or the control system are detachable from a breathable textile garment.

In another embodiment of a system of the invention, the textile extends partially or fully around the subject's torso or over one or both of the subject's shoulders.

In another embodiment of a system of the invention, the fabric material used is waterproof, hydrophobic, or bactericidal.

In another embodiment of a system of the invention, the fabric material is capable of being cleaned with soap and water without disturbing the electrical components of the wearable heart rhythm treatment device.

In another embodiment of a system of the invention, the control system is capable of receiving input from one or more secondary sensors that monitor additional information about the subject, including blood pressure, movement, and/or position.

In another embodiment of a system of the invention, the user interface can be worn on the wrist, ankle, or arm, handheld, or clipped to another article of clothing or a belt.

In another embodiment of a system of the invention, the user interface can detect proximity to the control system and emit a signal or alarm when too far separated from the control system.

In another embodiment of a system of the invention, a wearable heart rhythm treatment device comprises electrical and textile components organized in a balance and symmetrical manner and enclosed in a water resistant shell to minimize the impact of the components on the spine while erect and on the skin and musculature while reclining, wherein the separation and placement of the wearable heart rhythm device's component is designed to maximize the subject's comfort.

In another embodiment of a system of the invention, the control system is divided into at least two discrete components.

In another embodiment of a system of the invention, the components are spaced around the subject's body in some configuration so as to increase comfort.

In another embodiment of a system of the invention, the discrete components are operatively or communicatively connected to each other.

In another embodiment of a system of the invention, part of the control system is accessible and/or removable by the subject and the point of detachment/reattachment is designed to keep out water.

In another embodiment of a system of the invention, the control system is capable of receiving wirelessly transmitted signals.

In another embodiment of a system of the invention, one or more of the electrical conductors are coupled to the subject's skin by some adhesive mechanism.

In another embodiment of a system of the invention, one or more of the electrical conductors are coupled to the subject's skin by the exertion of pressure.

In another embodiment of a system of the invention, one or more of the electrical conductors are capable of continuously transmitting information wirelessly.

In another embodiment of a system of the invention, one or more of the electrical conductors are intended for use for an extended duration of at least three weeks.

In another embodiment of a system of the invention, one or more of the electrical conductors is made from a conductive textile material or woven into a breathable conductive fabric.

In another embodiment of a system of the invention, the electrical conductors are secured in place with one or more stabilizing elements to decrease noise.

In another embodiment of a system of the invention, one or more of the electrical conductors is made from a conductive ink substance that can be printed directly onto the subject's skin or onto a textile.

In another embodiment of a system of the invention, one or more of the electrical conductors comprises a bottom layer in contact with subject's skin that is adhesive and contains at least one conductive surface and a top layer that is not in direct contact with subject's skin that contains a controller and a battery.

In another embodiment of a system of the invention, the top layer is easily separable from the bottom layer by being pulled or slid apart.

In another embodiment of a system of the invention, one or more of the electrical conductors and/or the control system are fully enclosed between two layers of textile and not readily accessible to the subject.

In another embodiment of a system of the invention, one or more of the electrical conductors or the control system are attachable in a removable or unremovable way to a breathable textile garment.

In another embodiment of a system of the invention, a textile garment extends partially or fully around the subject's torso or over one or both of the subject's shoulders.

In another embodiment of a system of the invention, the textile garment comprises fabric material that is water-resistant, waterproof, hydrophobic, and/or bactericidal.

In another embodiment of a system of the invention, the fabric material is capable of being cleaned by being wiped with a wet cloth or paper towel.

In another embodiment of a system of the invention, the fabric material used is capable of exerting pressure.

In another embodiment of a system of the invention, the control system is capable of receiving input from one or more secondary sensors that monitor additional information about the subject, including heart rate, blood pressure or movement.

In another embodiment of a system of the invention, at least one separate user interface is communicatively coupled to the control system and capable of receiving and displaying information wirelessly.

In another embodiment of a system of the invention, a user interface can be worn on the wrist, ankle, or arm, handheld, or clipped to another article of clothing or a belt.

In another embodiment of a system of the invention, one or more electrical conductors contain an impedance-reduction mechanism, including but not limited to a gel release mechanism or a pressure-application mechanism.

In another embodiment of a system of the invention, a user interface can detect proximity to the control system and emit a signal or alarm when too far separated from the control system.

In another embodiment of a system of the invention, a user interface can be used by the subject to delay delivery of impending therapy.

In another embodiment of a system of the invention, a wearable heart rhythm controlling device is divided into at least two discrete components.

In another embodiment of a system of the invention, the control system is capable of advanced defibrillation techniques, including switching of shock vector, which refers to the path taken by the energy delivered through the body of the subject.

### BRIEF DESCRIPTION OF THE DRAWINGS

The invention can be better understood by referencing the following figures in which:

FIG. 1 is an anterior view of the textile garment of the invention holding all parts of the control system and some of the electrical conductors showing the location of the components within.

FIG. 2 is a posterior view of the garment of FIG. 1 showing the location of the components within.

FIG. 3 is an anterior view of the components within the garment of FIG. 1 with the garment hidden.

FIG. 4 is a view of a separate user interface useful according to the invention.

FIG. 5 is an exploded view of the electrical conductors responsible for sensing, that is, the sensing patch, according to the invention.

FIG. 6 is a schematic of a remote monitoring system contained within the wearable defibrillator that wirelessly communicates with a subcutaneous implantable cardiac monitor. Data can be analyzed and sent to the cloud for remote access via some smart device means.

FIG. 7 is picture depicting the wearable defibrillator vest of the invention on a subject.

FIG. 8 is chart comparing the features of the wearable defibrillator vest of the invention with the features of a Zoll LifeVest.

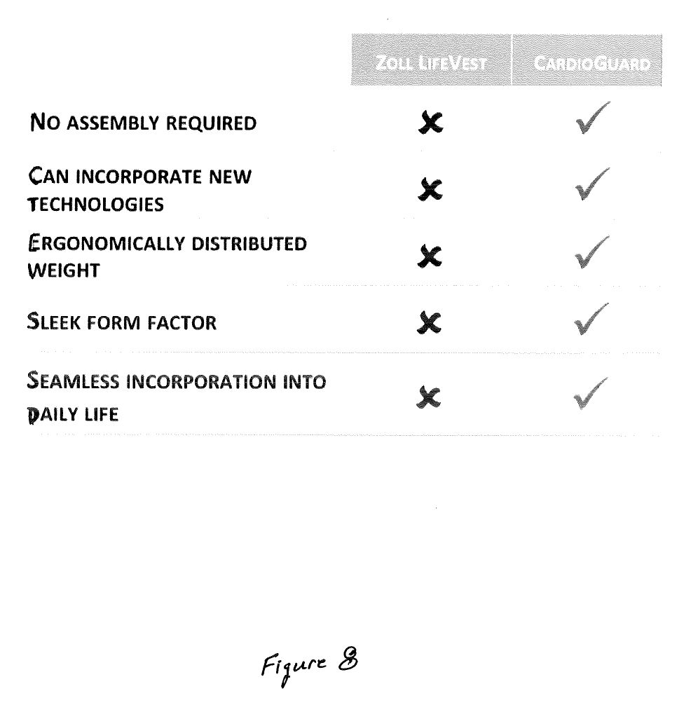

### DETAILED DESCRIPTION OF THE INVENTION

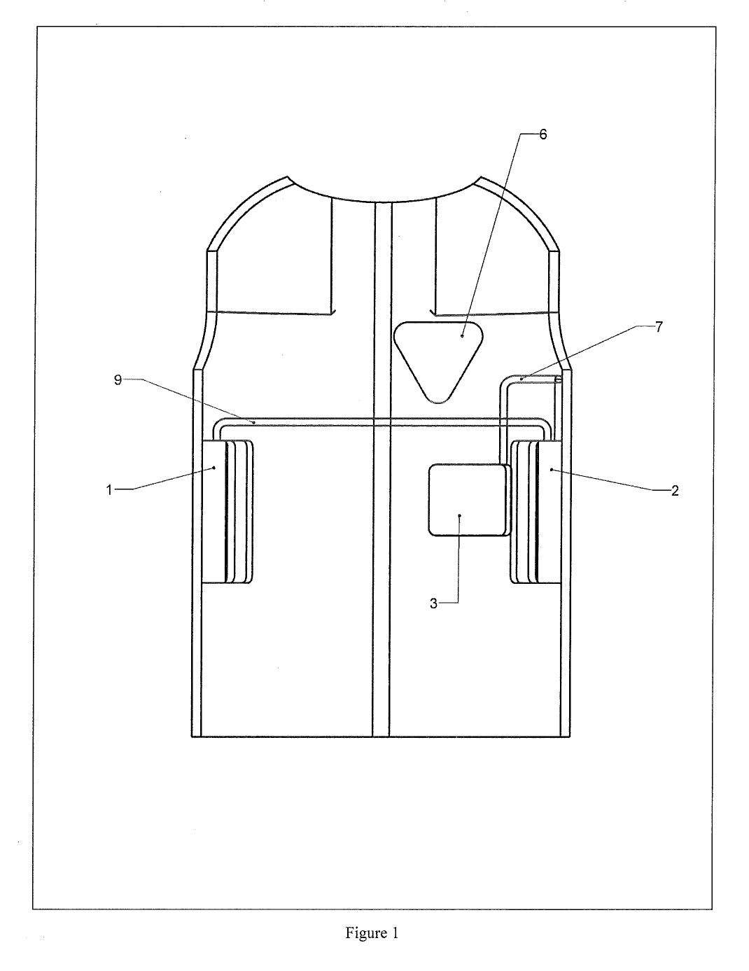

With reference to FIG. 1, there is displayed the anterior view of the garment as is worn with the location of the anterior placed devices shown. In this embodiment, the devices shown would not be visible or accessible and would be covered in another layer of fabric. It is seen that the control system is divided into two discrete components. A power source (1) is placed below the right arm, an electrical circuit and central processing unit (2) is placed below the left arm, and one of the electrical conductors responsible for defibrillator, hereby referred to as therapy pad(s) (3), is placed on the left side of the body below the chest and is connected to electrical circuit and central processing unit (2) via a wire (7). A sensing patch (6) is placed on the left side of the chest over the heart and is not directly connected to the garment. Power source (1) is electrically connected through wire or cable (9) to electrical circuit and central processing unit (2).

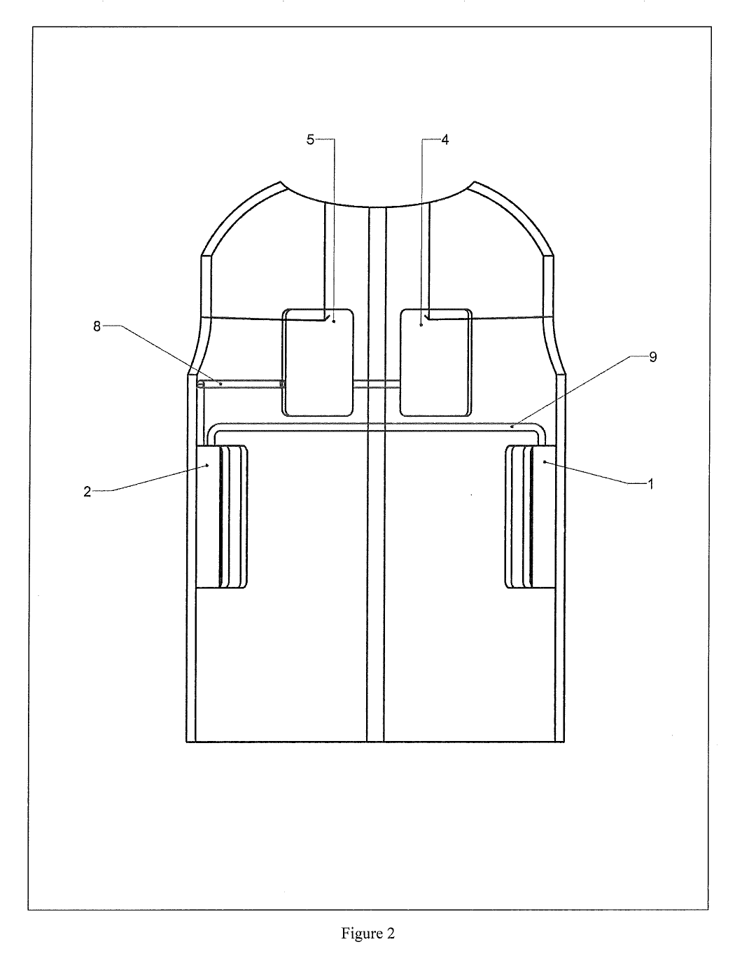

With reference to FIG. 2, there is displayed the posterior view of the garment as is worn with the location of the posterior placed devices shown. In this embodiment, the devices shown would not be visible or accessible and would be covered in another layer of fabric. Again, power source (1) is placed below the right arm, and central processing unit (2) is placed below the left arm. Two posterior therapy pads (4, 5) are placed along the upper back and are connected to electrical circuit and central processing unit (2) via a wire or cable (8). Power source (1) is connected to electrical circuit and central processing unit (2) to supply it with power via another wire or cable (9).

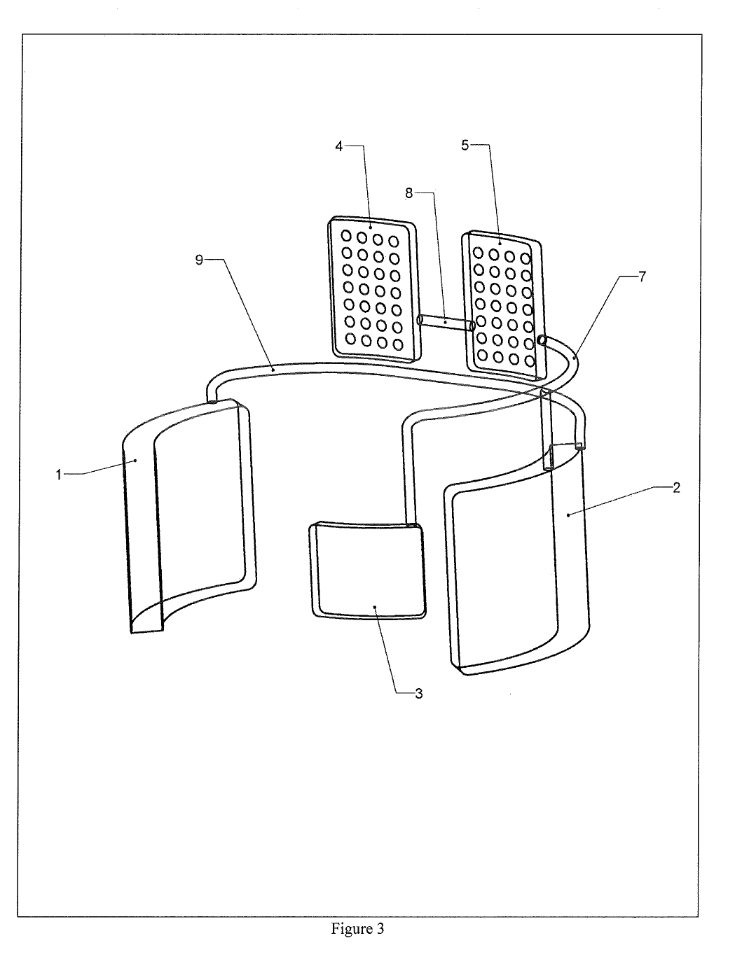

With reference to FIG. 3, the devices that are within the garment are displayed from an anterior view. The garment is hidden, or not shown, to better show the components. As is seen, power source (1) connects to electrical circuit and central processing unit (2) via wire or cable (9). Electrical circuit and central processing unit (2) powers two posterior therapy pads (4, 5) and the anterior therapy pad (3) via respective wires or cables (8, 7).

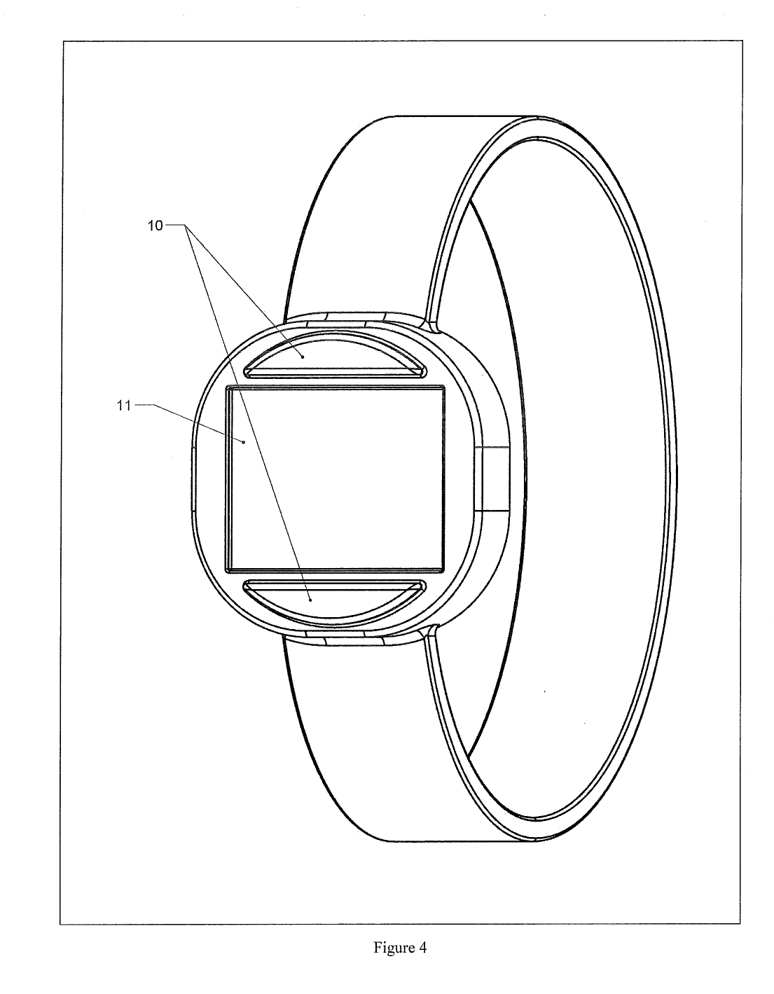

With reference to FIG. 4, a separate user interface is shown. The user interface is meant to be worn in the same manner as a watch is worn on the wrist and wirelessly transmits and receives data to and from central processing unit (2). Shown is an override mechanism (10), in which both buttons must be pressed to override impending therapy. There is also a screen (11) that shows relevant information to the user.

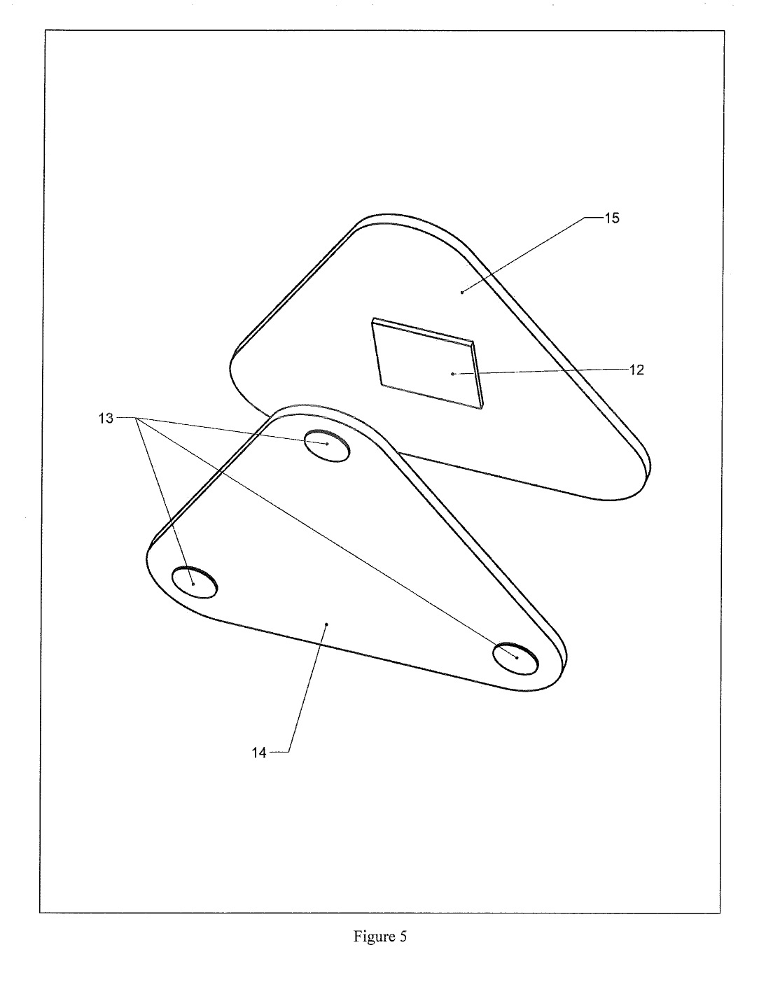

With reference to FIG. 5, there is displayed an exploded view of the sensing patch (6) that was referenced in FIG. 1. There is a bottom layer (14) that has adhesive to stick to the skin as well as three conductive surfaces (13). Bottom layer (14) can be separated from a top layer (15), which contains the technical components consisting of a battery and controller (12). In a preferred embodiment, technical components (12) would transmit ECG wirelessly to the central processing unit (2).

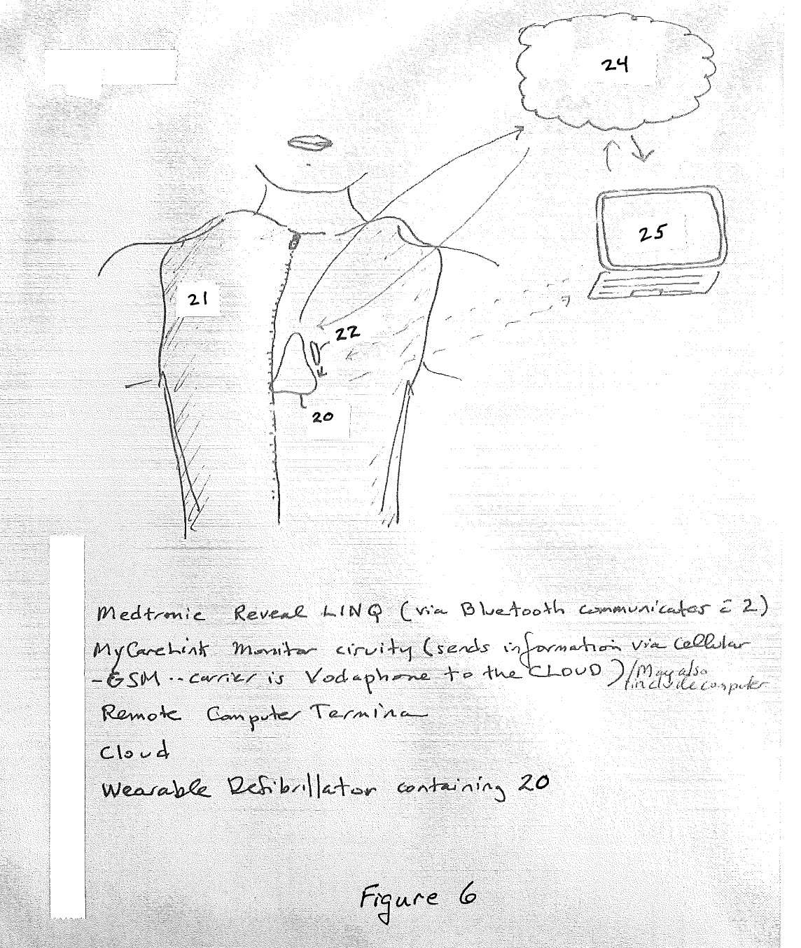

With reference to FIG. 6, there is a schematic of a remote monitoring system contained within the wearable defibrillator that wirelessly communicates with a subcutaneous implantable cardiac monitor. Data can be analyzed and sent to the cloud for remote access via some smart device means. Information gathered from (MyCareLink manufactured by Medtronic; 20) built into a wearable defibrillator (21). An implantable cardiac monitor (22) is inserted underneath the skin to record electrocardiographic signals. The data derived from these signals is sent wirelessly and securely to the remote monitoring system (20) via Bluetooth communication. This data can then be sent from the encased monitoring system contained within the wearable defibrillator via a wireless service (such as a GSM cellular service to the CLOUD (24). It then can be accessed by any remote webaccess/CLOUDACCESS method including computer terminal (25), smartphone, smart device, etc. Communication can be two-way and from remote computer terminal or device (25). The communication may not involve the cloud, but it will be secure and privacy protected.

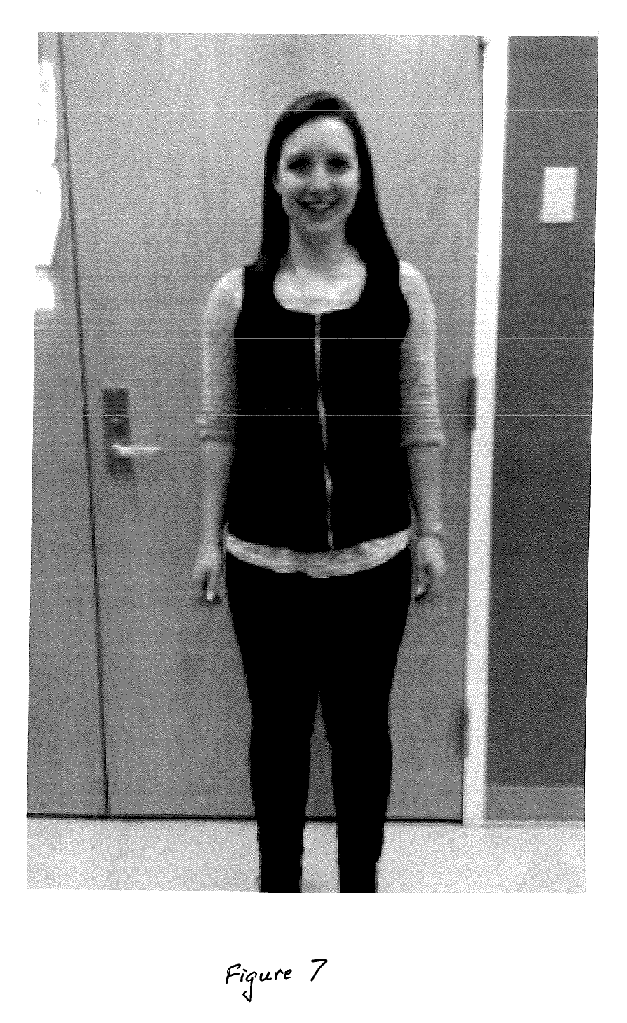

In a preferred embodiment of the invention, a wearable garment comprises the form of a shirt along with a separate user interface in the form of a watch worn on the wrist and a long-term sensing patch on the front of the body. See, FIG. 7. The wearable garment is made of a waterproof, breathable fabric capable of being cleaned by simply being wiped down with a moist paper towel or cloth (or rinsed with soap under the shower and towel dried). The control system is split up into two discrete components, which are separated on opposite lateral sides of the garment enclosed between two layers of fabric. The two components are operationally connected to each other with wires completely enclosed between layers of fabric as well. The back of the garment has two therapy pads with electrically conductive surfaces that are in contact with the patient's skin and another therapy pad laterally on the front left side of the garment. All therapy pads are under one layer of fabric and operationally connected to one component of the control system on one side, which is presumably capable of delivering the heart rhythm corrective therapy through the therapy pads.

The electrical conductors as a whole are capable of sensing and defibrillation. There may be separate conductors for each function. The preferred embodiment placing a single sensing patch containing three electrically conducting surfaces on the left upper chest for periods of about 4-6 weeks at a time before being replaced. In this preferred embodiment, the sensing patch has three conductive surfaces on the underside along with an adhesive securing it to the patient's skin. The bottom layer consists of these adhesive and conductive surfaces and the top layer consists of the battery and controller not in direct contact with the patient's skin. When ready for disposal, the top layer can be separated from the bottom layer by being pulled apart and then the bottom layer can be easily disposed while the components of the top layer are recycled. The sensing patch is capable of continuously transmitting information wirelessly to one of the two components of the control system in the garment. In alternative embodiments, the electrical conductors may be integrated directly into a textile garment by weaving or other method or held against skin by a pressure-related mechanism. The may also be made of a conductive ink material printed directly onto the skin or a garment.

In the preferred embodiment described above, there is also a separate user interface in the form of a watch that is capable of receiving and transmitting information wirelessly to the control system as well as sense proximity to the control system in the garment. When separated by too far a distance, an alarm is emitted. This user interface is capable of displaying information about battery life and any other pertinent information to the patient. In the event of impending therapy delivery, the user interface is also capable of emitting a visual, auditory, and tactile (in the form of vibration) alarm to alert the patient, and the patient can manually override the impending therapy by the press of a button on or near the user interface.

The sensing patch, garment, control system, and user interface work together in our current preferred embodiment to deliver effective therapy to patients when necessary in a way that optimizes their long-term comfort and compliance.

In another preferred embodiment, an implantable cardiac monitor such as the Medtronic LINQ is used for sensing the heart rhythm The LINQ is a heart rate and rhythm recording device that is about the size of a small paper clip. It is injected underneath the skin typically just to the left of the sternum. This type of heart monitor could transmit information wirelessly to the wearable defibrillator's self-contained computer which could analyze the signal/information and determine whether defibrillation therapy is required. If so, an alert can occur in the wearable defibrillator itself, or through the patient interface in the form of either and visual signal, audio signal, or vibration. The wearer of the defibrillator will have the opportunity to abort the delivery of therapy by touching the wrist worn patient interface. The LINQ currently communicates with the Medtronic MyCareLink remote monitoring system. Components of said system could be built into the wearable cardioverter defibrillator and/or its computer and contained within the vest. This computerized system can wirelessly communicate with a separate easily wearable watch like user interface and also send data wirelessly to a remote device (perhaps through a secure cloud service).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

We claim:

1. A wearable heart rhythm treatment system for treatment of a subject, which comprises:
a self-contained ergonomic waterproof or water-resistant, wearable heart rhythm treatment device comprising at least two therapy delivering electrical conductors coupled to the subject's skin for the purpose of treating an abnormal heart rhythm;
at least two sensing electrical conductors coupled to the subject's skin to detect abnormal heart rhythms;
a control and remote monitoring system comprising of at least one power source, at least one electrical circuit including a capacitor, and at least one computer/central processing unit capable of analyzing the electrical signal sensed by the above system and providing a heart rhythm corrective therapy through one or more of said electrical conductors to treat the heart rhythm abnormality; and
a user interface which wirelessly communicates with the subject and provides feedback as to the status of the subject, their heart rhythm, including alerts, alarms, and necessary actions in order to abort said therapy,
wherein all components of said wearable system are designed, constructed, and configured to minimize the weight of said system applied to the patient's shoulders and resultant lumbar spine compression while standing and component pressure on the abdomen or thorax while lying down or supine.

2. The system of claim 1, wherein the sensing system uses electrical conductors that are directly attached to the wearable heart rhythm treatment device.

3. The system of claim 2, wherein the electrical conductors are long-term adhesive electrodes.

4. The system of claim 1, wherein the sensing electrical conductors coupled to the skin are contained in a separate system subcutaneously implanted cardiac monitoring device, which is closely coupled to the wearable heart rhythm treatment device and communicates wirelessly heart rhythm related information from the subject.

5. The system of claim 1 in which the control system and remote monitoring system contained within the wearable heart rhythm treatment device can wirelessly and securely send and receive information to and/or from “the cloud” or other remote locations or sites regarding the wearable heart rhythm device's performance, function, arrhythmia detection and treatment, alerts, and alarms, which information is then accessible via a world wide web connected computerize system.

6. The system of claim 5 in which the remote monitoring system utilizes a cellular service, wife service, and/or other wireless system to transmit and receive data.

7. The system of claim 1, wherein the treatment therapies includes both cardioversion and defibrillation therapies.

8. The system of claim 7, wherein the treatment therapies also includes pacing therapies.

9. The system of claim 1, wherein detecting abnormal heart rhythms is categorized into significant slow heart rhythms requiring pacing therapy and significant fast heart rhythms requiring cardioversion or defibrillation therapy.

10. The system of claim 1, wherein the user interface is a separately wearable device that wirelessly displays and communicates information from the wearable heart rhythm device to the subject in order to provide information regarding their status as well as that of their wearable heart rhythm device, any arrhythmias and/or shock warnings or alerts, as well as the ability to abort a shock.

11. The system of claim 10, wherein said user interface is a wrist worn device similar to a watch.

12. A wearable treatment system for treatment of a subject, comprising:
an ergonomic waterproof or water-resistant wearable heart rhythm treatment device comprising at least two therapy delivering electrical conductors coupled to the subject's skin for the purpose of treating an abnormal heart rhythm;
at least two sensing electrical conductors coupled to the subject's skin for the purpose of detecting abnormal heart rhythms;
a control and remote monitoring system comprising of at least one power source, at least one electrical circuit including a capacitor, and at least one computer/central processing unit capable of analyzing the electrical signal sensed by the above system and providing a heart rhythm corrective therapy through one or more of said electrical conductors to treat the heart rhythm abnormality; and
a user interface which wirelessly communicates with the subject and provides feedback as to the status of the subject, their heart rhythm, including alerts, alarms, and necessary actions in order to abort said therapy,
wherein all components of said wearable system are designed, constructed, and configured in order to minimize the weight of said system applied to the patient's shoulders and resultant lumbar spine compression while standing; and component pressure on the abdomen or thorax while lying down or supine.

13. The system of claim 1, wherein part of the control system, sensing electrodes, and/or power supply are accessible and/or removable by the subject and the point of detachment/reattachment is designed to keep out water.

14. The system of claim 1, wherein one or more of the electrical conductors are made from a conductive textile material or woven into a breathable conductive fabric.

15. The system of claim 14, wherein the electrical conductors are secured in place with one or more stabilizing elements to decrease noise.

16. The system of claim 1, wherein one or more of the electrical conductors and/or the control system are fully enclosed between two layers of textile fabric and not readily accessible to the subject.

17. The system of claim 1, wherein one or more of the electrical conductors or the control system are detachable from a breathable textile garment.

18. The system of claim 17, wherein the textile garment extends partially or fully around the subject's torso or over one or both of the subject's shoulders.

19. The system of claim 18, wherein the textile garment comprises fabric material that is water-resistant, waterproof, hydrophobic, and/or bactericidal.

20. The system of claim 17, wherein the textile garment comprises fabric material capable of being cleaned with soap and water without disturbing the electrical components of the wearable heart rhythm treatment device.

21. The system of claim 1, wherein the control system is capable of receiving input from one or more secondary sensors that monitor additional information about the subject, including blood pressure, movement, and/or position.

22. The system of claim 10, wherein the user interface can be worn on the wrist, ankle, or atm, handheld, or clipped to another article of clothing or a belt.

23. The system of claim 22, wherein the user interface can detect proximity to the control system and emit a signal or alarm when too far separated from the control system.

24. A wearable heart rhythm treatment device or system comprised of electrical and textile components organized in a balanced and symmetrical manner and enclosed in a water-resistant or waterproof shell to minimize the impact of the components on the spine while erect and on the skin and musculature while reclining, wherein the separation and placement of the wearable heart rhythm device's components is designed to maximize the subject's comfort.

25. The wearable heart rhythm treatment device or system of claim 24 which is designed for ease of use, comfort, with minimal maintenance and assembly.

26. The wearable heart rhythm treatment device or system of claim 24 wherein the components are all contained within self-enclosed devices to minimize assembly and care.

27. The system of claim 1, wherein the wearable heart rhythm treatment device is comprised of two discrete elements to provide for separation/replacement/ and/or charging of the power supply, one of the elements being the wearable defibrillator and computer/remote monitor and the second element being the power supply or charging device.

28. The system of claim 12, wherein the wearable heart rhythm treatment device is comprised of two discrete elements to provide for separation/replacement/ and/or charging of the power supply, one of the elements being the wearable defibrillator and computer/remote monitor and the second element being the power supply or charging device.

29. The system of claim 1, wherein the power supply is charged via a separate plugin charger or an external inductance charger.

30. The system of claim 12, wherein the power supply is charged via a separate plugin charger or an external inductance charger.
